(19) United States
(12) Patent Application Publication
(10) Pub. No.: US 2011/0097405 A1
(43) Pub. Date: Apr. 28, 2011

(54) ESTRADIOL-CONTAINING DRUG DELIVERY SYSTEM

(75) Inventors: Adrian Funke, Berlin (DE); Sascha General, Berlin (DE); Ildikó Terebesi, Berlin (DE); Christian Zurth, Berlin (DE); Sofia Alincic-kunz, Berlin (DE); Matthias Schäfer, Berlin (DE); Thomas Holler, Berlin (DE); Konstanze Diefenbach, Berlin (DE)

(73) Assignee: Bayer Schering Pharma Aktiengesellschaft, Berlin (DE)

(21) Appl. No.: 12/867,409

(22) PCT Filed: Feb. 5, 2009
(86) PCT No.: PCT/EP09/51303

§ 371 Date: Jan. 4, 2011

Related U.S. Application Data
(60) Provisional application No. 61/028,302, filed on Feb. 13, 2008.

(30) Foreign Application Priority Data
Feb. 13, 2008 (EP) 08002633.9

(51) Int. Cl. A61K 31/567; A61K 9/00; A61K 31/56; A61P 19/10; A61P 25/24; A61P 1/08; A61P 25/06; A61P 5/24

(52) U.S. Cl. 424/485; 424/484; 514/182; 424/488; 424/486; 514/170

(57) ABSTRACT

The present invention relates to drug delivery systems in the form of thin water-soluble films (wafers), which contain estradiol, or derivatives thereof, in low amounts. The wafers of the present invention are suitable for treating, alleviating or preventing a physical condition in a female mammal caused by insufficient endogenous levels of estrogen.

### FIELD OF THE INVENTION

The present invention relates to drug delivery systems in the form of thin water-soluble films (wafers), which contain estradiol, or derivatives thereof, in low amounts. The wafers of the present invention are suitable for treating, alleviating or preventing a physical condition in a female mammal caused by insufficient endogenous levels of estrogen.

### BACKGROUND OF THE INVENTION

While drugs, such as estrogens, may be included in traditional standard oral tablet or capsule formulations to provide an accurate and consistent dose, such delivery forms have several disadvantages in both the administration and preparation of the drug. For example, it has been estimated that about 50% of the population have problems swallowing tablets (see Seager in J. Pharmacol. Pharm. 1998; 50; 375-382), and patients such as children or the elderly who will not, or cannot, swallow tablets or capsules represent a challenge for the pharmaceutical industry. The pharmaceutical industry has tried to meet this challenge by developing a number of different drug delivery systems, including rapid in-mouth disintegrating tablets, tablets which disintegrate in liquid prior to ingestion, liquids and syrups, gums and even transdermal patches. However, each of these drug delivery systems can pose their own problems.

Transdermal patches can be inconvenient and uncomfortable as well as rather expensive to produce. Furthermore, the drug flux through the skin can also raise very complex dosing issues. Liquids are particularly useful for children. However, liquids can be inconvenient for adults and can be relatively expensive to formulate, package and transport. Tablets that can be dissolved in a liquid before ingestion can also be useful. However, they can also be quite inconvenient in that they require liquid and a drinking container to be provided. Furthermore, time is required for disintegration and/or dissolution, even when effervescent tablets are used. Finally, these drug delivery systems can be quite messy as they typically leave a particulate and/or scum in the glass. Rapid in-mouth disintegrating tablets, such as chewable or self disintegrating tablets offer great convenience. However, chewable or self-disintegrating tablets often present real taste masking problems as the act of chewing can disrupt protective coatings. Furthermore, chewable or self-disintegrating tablets are often associated with an unpleasant mouthfeel. Moreover, the fear of swallowing, chewing, or choking on such solid shaped articles is still a concern in certain populations. In addition, the fragility/5 friability of such porous, and low-pressure molded tablets makes them difficult to carry, store, handle and administer to patients, especially the children and the elderly.

Thus, there is a need for reliable delivery systems with improved patient compliance, i.e. where the dosing is easy and allows the patients to take their medications discretely wherever and whenever needed. Water-soluble films (wafers) fulfil those criteria. Usually, such wafers dissolve quickly in the saliva present in the mouth thereby releasing the active ingredient(s) which, in turn, can then be absorbed via the lingual, sublingual, buccal and/or the palatal route.

The pharmaceutical industry is constantly aiming at improving delivery systems in order to make a better utilisation of a given drug dose. Stated differently, there is a constant need for delivery systems where the drug load can be lowered while, at the same time, still give rise to clinical relevant concentrations of the drug in the blood stream. This is particularly relevant when high-potent drugs, such as steroid hormones, are to be administered. Lowering the dose of, e.g., a steroid hormone while still obtaining clinical relevant concentrations of the steroid hormone in the blood stream not only allows for savings in the pharmaceutical industry, as smaller amounts of drug is needed, but also allows for smaller total amounts of the steroid hormone to be administered to the patients. Evidently, this may lead to fewer cases of overdosing and less pronounced side-effects. The importance of administration of low doses of steroid hormones, such as estrogens, is also emphasised in the FDA guidelines where sponsors are encouraged to investigate dosing schedules and drug delivery systems that can achieve efficacy with the lowest possible exposures (Guidance for Industry: Estrogen and Estrogen/Progestin Drug Products to Treat Vasomotor Symptoms and Vulvar and Vaginal Atrophy Symptoms—Recommendations for Clinical Evaluation; U.S. Department of Health and Human Services; Food and Drug Administration; CDER; January 2003).

Yet another aim when creating new dosage forms for highly active drugs is to achieve low inter-individual variability with respect to the resulting serum levels of the drug in different patients in order to guarantee that comparable dosages of the drug will lead to comparable effects in different patients. Low inter-individual variability with respect to the resulting estradiol level and the resulting estrogenic effect, respectively, allow for administration of the same lowest effective dose in a high number of patients.

The present inventors have surprisingly realised that a unit dosage form (in the form of a wafer) that contains a low dose of estradiol can be administered to female mammals via the intraoral route and still give rise to clinical relevant concentrations in the blood stream.

Low dose administration of estradiol via the nasal route has been described by Dören et al. Maturitas 2001; 38; 23-30 and Dooley et al. Drugs 2001; 61; 2243-2262, and a nasal spray containing estradiol (Aerodiol®) has previously been marketed. Intranasal administration of 300 μg/day of estradiol has been shown to be as efficient as standard oral therapy, i.e. as oral administration of 2 mg/day of estradiol, but at the same time showing reduced side effects, i.e. it is better tolerated gynecologically. More particularly, fewer incidences of mastalgia and withdrawal bleedings have been reported for patients receiving (low dose) intranasally administered estradiol as compared to standard oral therapy, cf. Mattson, Climateric 2002; 5 (Suppl. 2); 40-45. Furthermore, beneficial effects on some lipid parameters, i.e. favourable action on the endogenous levels of lipoprotein (a), apolipoprotein B, total cholesterol and low-density lipoprotein cholesterol, and on markers of bone resorption, bone formation and bone mineral density in some postmenopausal women have been described (Palacios, Climateric 2002; 5 (Suppl. 2); 32-39). Furthermore, in comparison to standard oral treatment, intranasal administration of estradiol showed a significantly lower rate of tumour induction and reduced tumour growth (Mattson, Climateric 2002; 5 (Suppl. 2); 40-45). Moreover, a lower incidence of breast tenderness was found with intranasal administration of estradiol as compared to standard oral treatment.

The pharmacokinetics of intranasally absorbed estradiol differs from that of estradiol taken orally in that the uptake is very rapid; maximum plasma levels are achieved within 10-30 minutes and the levels return to 10% of the peak value about two hours after administration, and reach the pre-treatment level about 12 hours after administration (Al-Azzawi, Climateric 2002; 5 (Suppl. 2); 27-31). As a consequence, daily intranasal administration of estradiol results in a “puls-like” profile that is significantly different from the sustained profile observed after oral and transdermal administration. Despite this “puls-like” profile, intranasal administration of low amounts of estradiol has turned out to be as efficient as standard oral therapy and, as a further benefit, causing fewer side effects.

However, one main problem by intranasal administration is the highly inconvenient administration form. Nasal administration, may give rise to local side effects, such as itching, rhinorrhoea, sneezing and nosebleeds, and the patient compliance is generally considered to be poor by this form of administration.

The present inventors have discovered that a “puls-like” absorption of estradiol can be achieved by the unit dosage form of the invention. Thus, the beneficial properties, i.e. reduction of side effects, obtained by nasal administration of estradiol, are also obtained by administration of the unit dosage form of the invention. Furthermore, the administered dose of estradiol can be lowered significantly compared to oral administration. The lowered dose of estradiol is particularly beneficial as opposed treatment (continuous or cyclic co-administration of a progestin) is not an absolute requirement because the endometrium does not proliferate upon administration of such low doses of estradiol.

Accordingly, it is an object of the invention to provide a unit dosage form to be applied to the oral cavity, which provides the same beneficial therapeutic properties as does intranasal administration, but where the patient compliance is higher.

Another object of the invention is to provide a unit dosage form to be applied to the oral cavity, which gives rise to a “puls-like” pharmacokinetic profile of estradiol.

Yet another object of the invention is to provide a unit dosage form to be applied to the oral cavity, where an increased bioavailability, as compared to intranasal administration, is obtained.

Still another object of the invention is to provide a unit dosage form which contains a lowered dose of estradiol (or derivatives thereof), as compared to standard oral treatment, but which still give rise to a clinical relevant concentration of estradiol in the blood stream of the patient.

A further object of the invention is to provide a unit dosage form which contains a lowered dose of estradiol (or derivatives thereof), as compared to standard intranasal treatment, but which still give rise to a clinical relevant concentration of estradiol in the blood stream of the patient.

A still further object of the invention is to provide a unit dosage form to be applied to the oral cavity which gives rise to fewer side effects, as compared to standard oral treatment, but which is still effective in treating, alleviating or preventing a physical condition in a female mammal caused by insufficient endogenous levels of estrogen.

An even further object of the invention to provide a unit dosage form to be applied to the oral cavity which is better tolerated gynecologically, as compared to standard oral treatment, but which is still effective in treating, alleviating or preventing a physical condition in a female mammal caused by insufficient endogenous levels of estrogen.

Chewable taste-masked pharmaceutical compositions are described in U.S. Pat. No. 4,800,087.
Taste-masked orally disintegrating tablets (ODTs) are described in US 2006/0105038.
Taste-masking coating systems are described in WO 00/30617.
Buccal tablets comprising estradiol are disclosed in EP 0 371 466.
Medicated papers for oral transmucosal administration are described in EP 1 867 321.
Taste-masked wafers are described in WO 03/030883.
Steroid-containing wafers are described in US 2003/0068378, US 2007/0292479 and US 2006/0222708.

### SUMMARY OF THE INVENTION

In a first aspect, the present invention relates to a unit dosage form comprising a thin water-soluble film matrix, wherein
a) said film matrix comprises at least one water-soluble matrix polymer;
b) said film matrix comprises 10-200 μg of estradiol, or a therapeutically equivalent amount of a hydrate of estradiol or a therapeutically equivalent amount of a pharmaceutically acceptable ester of estradiol; and
c) said film matrix has a thickness of less than 300 μm.

In another aspect, the present invention relates to a unit dosage form of the invention for use as a medicament.

In a further aspect, the present invention relates to a unit dosage form of the invention for treating, alleviating or preventing a physical condition in a female mammal caused by insufficient endogenous levels of estrogen. Examples of such physical conditions include, but is not limited to, osteoporosis, headaches, nausea, depression, vasomotor symptoms, symptoms of urogenital atrophy, decrease in bone mineral density, and increased risk or incidence of bone fracture.

Other aspects of the present invention will be apparent from the below description and the appended claims.

### DETAILED DESCRIPTION OF THE INVENTION

Herein, the term “estradiol” is intended to mean that the estradiol may be in the form of 17-α-estradiol or 17-β-estradiol. Preferably, the estradiol is in the form of 17-β-estradiol. The term “estradiol” also covers hydrated forms of estradiol, in particular estradiol hemihydrate.

When used herein, the term “pharmaceutically acceptable ester of estradiol” refers to those esters of estradiol which would be apparent to the pharmaceutical chemist, i.e. those which are substantially non-toxic and which may favourably affect the pharmacokinetic properties of estradiol, such as palatability, absorption, distribution, metabolism and excretion. Typically, an ester of estradiol is in the 3-position or 17-position of estradiol. Specific examples of pharmaceutically acceptable esters of estradiol include estradiol valerate, estradiol acetate, estradiol propionate, estradiol enantate, estradiol undecylate, estradiol benzoate, estradiol cypionate, estradiol sulfate and estradiol sulfamate.

In the present context, the terms “estradiol derivatives” and “derivatives of estradiol” are intended to cover hydrated forms of estradiol and pharmaceutically acceptable esters of estradiol. Although the various estradiol derivates (such as the hemihydrate and the pharmaceutically acceptable esters) may not be explicitly cited in connection with each and every embodiment of the invention, it will be understood that whenever statements concerning “estradiol” are made herein, such statements also apply to the hemihydrate form of estradiol as well as to the pharmaceutically acceptable esters of estradiol.

The term “water-soluble film matrix”, wherein used herein, refers to a thin film which comprises, or consists of, a water-soluble polymer and estradiol as well as other auxiliary components dissolved or dispersed in the water-soluble polymer. In a preferred embodiment, at least the estradiol is completely dissolved in the water-soluble polymer.

As used herein, the term “water-soluble polymer” refers to a polymer that is at least partially soluble in water, and preferably fully or predominantly soluble in water, or absorbs water. Polymers that absorb water are often referred to as being “water-swellable polymers”. The materials useful for the present invention may be water-soluble or water-swellable at room temperature (about 20° C.) and other temperatures, such as temperatures exceeding room temperature. Moreover, the materials may be water-soluble or water-swellable at pressures less than atmospheric pressure. Desirably, the water-soluble polymers are water-soluble, or water-swellable having at least 20% by weight water uptake. Water-swellable polymers having 25% by weight, or more, water uptake, are also useful. The unit dosage forms of the present invention formed from such water-soluble polymers are desirably sufficiently water-soluble to be dissolvable upon contact with bodily fluids, in particular saliva. In a preferred embodiment of the invention, the water-soluble polymer is a mucoadhesive polymer. This will allow for transmucosal delivery of the estradiol and ensure efficient uptake of the molecule by avoiding the first pass metabolism. The water-soluble polymer typically constitutes from 50-99.9% by weight, such as from 75-99% by weight, of the water-soluble film matrix.

The water-soluble matrix polymer (constituting the major part of the water-soluble film matrix) can be selected from the group consisting of a cellulosic material, a synthetic polymer, a gum, a protein, a starch, a glucan and mixtures thereof.

Examples of cellulosic materials suitable for the purposes described herein include carboxymethyl cellulose, methyl cellulose, ethyl cellulose, hydroxymethyl cellulose, hydroxyethyl cellulose, hydroxypropyl cellulose, hydroxymethylpropyl cellulose, hydroxypropylmethyl cellulose and combinations thereof. Particularly preferred cellulosic materials are hydroxypropylmethyl cellulose and hydroxypropyl cellulose, in particular hydroxypropylmethyl cellulose.

Examples of synthetic polymers include polymers commonly used as immediate-release (IR) coatings for pharmaceuticals, such as the PVA-PEG co-polymers, which are commercially available in different grades under the trademark Kollicoat® IR. Further examples of synthetic polymers include polyacrylic acid and polyacrylic acid derivatives

Examples of water-soluble gums include gum arable, xanthan gum, tragacanth, acacia, carageenan, guar gum, locust bean gum, pectin, alginates and combinations thereof.

Useful water-soluble protein polymers include gelatine, zein, gluten, soy protein, soy protein isolate, whey protein, whey protein isolate, casein, levin, collagen and combinations thereof.

Examples of useful starches include gelatinised, modified or unmodified starches. The source of the starches may vary and include pullulan, tapioca, rice, corn, potato, wheat and combinations thereof.

Additional water-soluble polymers, which may be used in accordance with the present invention, include dextrin, dextran and combinations thereof, as well as chitin, chitosin and combinations thereof, polydextrose and fructose oligomers.

In one embodiment of the invention the estradiol is anhydrous estradiol or estradiol hemihydrate, preferably estradiol hemihydrate.

In another embodiment of the invention a pharmaceutically acceptable ester of estradiol is incorporated in the film matrix. Specific examples of such pharmaceutically acceptable esters of estradiol include estradiol valerate, estradiol acetate, estradiol propionate, estradiol enantate, estradiol undecylate, estradiol benzoate, estradiol cypionate, estradiol sulfate, and estradiol suifamate. In an interesting embodiment of the invention, the ester of estradiol is estradiol valerate.

The unit dosage form of the invention may, in certain embodiments comprise a dose of estradiol in the range of 5-1000 μg of estradiol, such as 10-750 μg of estradiol, e.g. 25-500 μg of estradiol. However, as will be understood from the present disclosure, the film matrix typically comprises 10-200 μg of estradiol, such as 10-60 μg of estradiol or >60-200 μg of estradiol.

In a preferred embodiment of the invention, the film matrix comprises an “ultra-low” dose of estradiol, i.e. a dose of 10-60 μg of estradiol, such as 25-60 μg of estradiol, preferably 30-50 μg of estradiol, more preferably 40-50 μg of estradiol, e.g. 40, 45, 46 or 50 μg of estradiol. Alternatively, the “ultra-low” dose of estradiol is 10-60 μg of estradiol, such as 10-50 μg of estradiol, preferably 20-40 μg of estradiol, more preferably 25-35 μg of estradiol, e.g. about 30 μg of estradiol.

In another preferred embodiment of the invention, the film matrix comprises a “very low” dose of estradiol, i.e. a dose of >60-200 μg of estradiol, such as 7°-160 μg of estradiol, e.g. 70-150 μg of estradiol, preferably 80-150 μg of estradiol, such as 80-120 μg or 120-150 μg of estradiol. Specific and preferred estradiol doses include 80, 85, 90, 100, 115, 120, 150 and 160 μg estradiol.

In yet another embodiment of the invention, the film matrix comprises a “medium low” dose of estradiol, i.e. a dose of >200-500 μg of estradiol, such as 250-300 μg of estradiol, e.g. 260-280 μg of estradiol, more preferably 265-275 μg of estradiol, e.g. about 270 μg of estradiol.

In still another embodiment of the invention, the film matrix comprises a “low” dose of estradiol, i.e. a dose of >5004000 of estradiol, such as >500-750 μg of estradiol.

Specific examples of doses of estradiol which may be comprised in the film matrix includes doses of about 10, 12.5, 15, 20, 30, 40, 45, 46, 50, 60, 70, 80, 85, 90, 100, 115, 120, 150, 160, 180, 200 or 270 μg of estradiol.

The above-mentioned doses preferably correspond to the daily dose. It should be understood that the above-mentioned doses are indicated with respect to anhydrous estradiol. If a hydrate of estradiol, such as estradiol hemihydrate, or a pharmaceutically acceptable ester of estradiol, such as estradiol valerate, is employed it will be understood that a dose which is therapeutically equivalent to the stated dose of anhydrous estradiol should be used. It is routine for those skilled in the art to determine pharmacologically/therapeutically equivalent doses of such other forms when the effective dose of anhydrous estradiol is known. Stated differently, if a hydrate of estradiol, such as estradiol hemihydrate, or a pharmaceutically acceptable ester of estradiol, such as estradiol valerate, is employed it will be understood that a dose which is equimolar to the stated dose of anhydrous estradiol should be used, provided that the absorption of estradiol and the derivative thereof is the same, cf. below. Thus, a “therapeutically equivalent amount of estradiol derivative X” can be calculated by the following formula:

Dose_{anhydrous estradiol}×(MW_{estradiol derivative X}/MW_{anhydrous estradiol})

where MW indicates the molecular weight of the estradiol compound in question. It will be understood that all of the above-indicated intervals and doses of estradiol (based on estradiol in anhydrous form) should be converted to the corresponding intervals and doses (using the above formula) if estradiol is not used in its anhydrous form. By way of example, and by application of the above formula, it can easily be calculated that a dose of 5-1000 μg of anhydrous estradiol corresponds to a dose of 5.2-1033 μg of estradiol hemihydrate. It will be understood, however, that the above formula can only be applied if the bioavailability and the Area Under the Curve (AUC) are identical for anhydrous estradiol and the derivative in question. Thus, if the absorption of the estradiol derivative in question differs from the absorption of anhydrous estradiol, the amount of the estradiol derivative required to achieve the plasma level of a given dose of anhydrous estradiol is decisive for determining the therapeutically equivalent amount.

Also, the paper of Timmer and Geurts provides guidance of how equivalent doses may be determined (see “Bioequivalence assessment of three different estradiol formulations in postmenopausal women in an open, randomized, single-dose, 3-way cross-over” in European Journal of Drug Metabolism and Pharmacokinetics, 24(1):47-53, 1999).

The unit dosage form of the invention is most preferably in the form of a thin film, which dissolves fast mainly due to the large surface area of the film, which wets quickly when exposed to the moist oral environment. Contrary to fast-dissolving tablets, which are usually soft, friable and/or brittle, the film is solid and strong, but still flexible. As indicated above, the film is thin and can be carried in the patient's pocket, wallet or pocket book.

The film may be applied under or on the tongue, to the upper palatine, to the inner cheeks or any oral mucosal tissue, of the female mammal. The film may be rectangular, oval, circular, or, if desired, a specific shape, cut to the shape of the tongue, the palatine or the inner cheeks, may be applied. The film is rapidly hydrated and will adhere onto the site of application. It then rapidly disintegrates and dissolves to release the estradiol for oralmucosal absorption.

Concerning the dimensions of the unit dosage form of the invention, the water-soluble film forming matrix is formed into a dry film which has a thickness of less than 300 μm, in particular less than 250 μm, preferably less then 200 μm, such as less than 150 μm. More preferably, the thickness is less than 125 μm, such as less than 100 μm. Stated differently, the thickness is typically in the range of from 10-300 μm, in particular in the range of from 15-250 μm, preferably in the range of from 20-200 μm, such as in the range of from 25-150 μm. More preferably, the thickness is in the range of from 25-125 μm, such as in the range of from 25-100 μm, e.g. in the range of from 30-90 μm, in particular in the range of from 40-80 μm. Specific, and preferred, examples include thicknesses of about 30 μm, about 40 μm, about 50 μm, about 60 μm, about 70 μm, about 80 μm, about 90 μm or about 100 μm. Specific, and particularly preferred, examples include thicknesses of about 40 μm, about 50 μm, about 60 μm, about 70 μm or about 80 μm.

The surface dimension (surface area) of the film matrix is typically in the range of from 2-10 cm², such as in the range of from 3-9 cm², e.g. in the range of from 3-8 cm², more preferably in the range of from 3-7 cm², in particular in the range of from 4-6 cm². Specific, and preferred, examples of the surface area include surface areas of about 3, 3.5, 4, 4.5, 5, 5.5, 6, 6.5 or 7 cm². Most preferably, the surface area is about 4, 4.5, 5, 5.5 or 6 cm².

The total weight of the film matrix will typically be in the range of from 5-200 mg, such as in the range of from 5-150 mg, e.g. in the range of from 10-100 mg. More preferably, the total weight of the film matrix is in the range of from 10-75 mg, such as in the range of from 10-50 mg. Specific, and preferred, examples of the weight of the film matrix include weights of about 15 mg, about 20 mg, about 25 mg, about 30 mg, about 40 mg or about 50 mg.

In another embodiment of the invention, the unit dosage form further comprises an absorption enhancer. Absorption enhancers have demonstrated their effectiveness in delivering e.g. high molecular weight drugs, such as peptides, that generally exhibit low buccal absorption rates. Such absorption enhancers may act by a number of mechanisms, such a increasing the fluidity of the cell membrane, extracting inter/intracellular lipids, altering cellular proteins or altering surface mucin. The most commonly used absorption enhancers include azone, fatty acids, bile salts and surfactants, such as sodium dodecyl sulfate. Specific examples of absorption enhancers include, but are not limited to, 2,3-lauryl ether, aprotinin, azone, benzalkonium chloride, cetylpyridinium chloride, cetyltrimethyl ammonium bromide, cyclodextrin, dextran sulfate, glycol, lauric acid, lysophosphatidylcholine, menthol, phosphatidylcholine, polyoxyethylene, polysorbate 80, polyoxyethylene, phosphatidylcholine, sodium EDTA, sodium glycocholate, sodium glycodeoxycholate, sodium lauryl sulfate, sodium dodecyl sulfate, sodium salicylate, sodium taurocholate and sodium taurodeoxycholate, sulfoxides. If incorporated, the absorption enhancer is typically present in the film matrix in an amount corresponding to 0.1-50% by weight of the film matrix, such as 1-20% by weight of the film matrix, e.g. 1-10% by weight of the film matrix. The absorption enhancer is typically comprised in the film matrix, i.e. the absorption enhancer is typically dissolved or dispersed in the film matrix. However, in a preferred embodiment of the invention, the unit dosage form does not contain an absorption enhancer.

Furthermore, the estradiol may be complexed with a cyclodextrin or a cyclodextrin derivative, such as those described in paragraph 0015 of US 2007/0292479. However, in a preferred embodiment of the invention, the estradiol is present in the unit dosage form in non-complexed form.

In addition to the water-soluble matrix polymer and estradiol, the unit dosage form of the invention may include a variety of various auxiliary components, such as taste-masking agents; organoleptic agents, such as sweeteners and flavours, anti- and de-foaming agents; plasticizing agents; surfactants; emulsifying agents; thickening agents; binding agents; cooling agents; saliva-stimulating agents, such as menthol; antimicrobial agents; colorants; etc. Such various auxiliary components are comprised in the film matrix and is typically dissolved or dispersed in the film matrix.

Suitable sweeteners include both natural and artificial sweeteners. Specific examples of suitable sweeteners include, e.g.:

a) water-soluble sweetening agents such as sugar alcohols, monosaccharides, disaccharides, oligosaccharides and polysaccharides such as maltit, xylit, mannit, sorbit, xylose, ribose, glucose (dextrose), mannose, galactose, fructose (levulose), sucrose (sugar), maltose, invert sugar (a Mixture of fructose and glucose derived from sucrose), partially hydrolyzed starch, corn syrup solids, dihydrochalcones, monellin, steviosides, and glycyrrhizin;
b) water-soluble artificial sweeteners such as the soluble saccharin salts, i.e., sodium or calcium saccharin salts, cyclamate salts, the sodium, ammonium or calcium salt of 3,4-dihydro-6-methyl-1,2,3-oxathiazine-4-one-2,2-dioxide, the potassium salt of 3,4-dihydro-6-methyl-1,2,3-oxathiazine-4-one-2,2-dioxide (acesulfame-K), the free acid form of saccharin, and the like;
c) dipeptide-based sweeteners, such as L-aspartic acid derived sweeteners, such as L-aspartyl-L-phenylalanine methyl ester (aspartame), L-alpha-aspartyl-N-(2,2,4,4 5 tetramethyl-3-thietanyl)-D-alaninamide hydrate, methyl esters of L-aspartyl-L phenylglycerin and L-aspartyl-L-2,5, dihydrophenylglycine, L-aspartyl-2,5-dihydro-L phenylalanine, L-aspartyl-L-(1-cyclohexyen)-alanine, and the like;
d) water-soluble sweeteners derived from naturally occurring water-soluble sweeteners, such as a chlorinated derivatives of ordinary sugar (sucrose), known, for example, under the product description of sucralose®; and
e) protein-based sweeteners such as thaurnatoccous danielli (Thaurnatin I and II).

In general, an effective amount of sweetener is utilised to provide the level of sweetness desired for a particular composition, and this amount will vary with the sweetener selected. This amount will normally be from about 0.01% to about 20% by weight, preferably from about 0.05% to about 10% by weight, of the film matrix. These amounts may be used to achieve a desired level of sweetness independent from the flavour level achieved from any optional flavour oils used.

Useful flavours (or flavouring agents) include natural and artificial flavours. These flavourings may be chosen from synthetic flavour oils and flavouring aromatics, and/or oils, oleo resins and extracts derived from plants, leaves, flowers, fruits and so forth, and combinations thereof. Non-limiting examples of flavour oils include: spearmint oil, cinnamon oil, peppermint oil, clove oil, bay oil, thyme oil, cedar leaf oil, oil of nutmeg, oil of sage, and oil of bitter almonds. Also useful are artificial, natural or synthetic fruit flavours such as vanilla, chocolate, coffee, cocoa and citrus oil, including lemon, orange, grape, lime and grapefruit, and fruit essences including apple, pear, peach, strawberry, raspberry, cherry, plum, pineapple, apricot and the like. These flavourings can be used individually or in combination. Commonly used flavours include mints such as peppermint, artificial vanilla, cinnamon derivatives, and various fruit flavours, whether employed individually or in combination. Flavourings such as aldehydes and esters including cinnamyiacetate, cinnamaldehyde, citral, diethylacetal, dihydrocarvyl acetate, eugenyl formate, p-methylanisole, and the like may also be used. Further examples of aldehyde flavourings include, but are not limited to acetaldehyde (apple); benzaldehyde (cherry, almond); cinnamicaldehyde (cinnamon); citral, i.e., alpha citral (lemon, lime); neral, i.e. beta citral (lemon, lime); decanal (orange, lemon); ethyl vanillin (vanilla, cream); heliotropine, i.e., piperonal (vanilla, cream); vanillin (vanilla, cream); alpha-amyl cinnamaldehyde (spicy fruity flavours); butyraldehyde (butter, cheese); valeraldehyde (butter, cheese); citronellal (modified, many types); decanal (citrus fruits); aldehyde C-8 (citrus fruits); aldehyde C-9 (citrus fruits); aldehyde C-12 (citrus fruits); 2-ethyl butyraldehyde (berry fruits); hexenal, i.e. trans-2 (berry fruits); tolyl aldehyde (cherry, almond); veratraldehyde (vanilla); 12,6-dimethyl-5-heptenal, i.e. melonal (melon); 2-dimethyloctanal (greenfruit); and 2-dodecenal (citrus, mandarin); cherry; grape; essential oils, like menthol; mixtures thereof; and the like.

The amount of flavouring employed is normally a matter of preference, subject to such factors as flavour type, individual flavour, and strength desired. The amount may be varied in order to obtain the result desired in the final product. Such variations are within the capabilities of those skilled in the art without the need for undue experimentation. In general, amounts from about 0.01% to about 10% by weight of the film matrix are employed.

As discussed above, the unit dosage form may also include an anti-foaming and/or de-foaming agent, such as simethicone, which is a combination of a polymethylsiloxane and silicon dioxide. Simethicone acts as either an anti-foaming or de-foaming agent which reduces or eliminates air from the film composition. Anti-foaming agents will aid in preventing the introduction of air into the composition, while de-foaming agents will aid removing air from the composition.

The unit dosage form may be prepared and adhered to a second layer, i.e. a support or backing layer (liner) from which it is removed prior to use, i.e. before being introduced into the oral cavity. Preferably, the support or backing material is not water-soluble and may preferably consist of polyethylene-terephthalate, or other suitable materials well known to the skilled person. If an adhesive is used, it should preferably be a food grade adhesive that is not ingestible and does not alter the properties of the active ingredient(s).

In another embodiment of the invention, the unit dosage form of the invention comprises a further active drug substance, such as a progestin. This further active drug substance is typically comprised in the film matrix.

It should be understood, however, that in an interesting embodiment of the invention, the unit dosage form of the invention does not contain a progestin. Accordingly, in another interesting embodiment of the invention, estradiol (or a hydrate of estradiol or a pharmaceutically acceptable ester of estradiol) is the only or sole therapeutically active drug substance present in the unit dosage form.

While the present disclosure is mainly concerned with wafers containing estradiol, or derivatives thereof, it is contemplated that the invention can also be practiced with other compounds exhibiting estrogenic activity, such as ethinylestradiol, estrone, mestranol, estriol, estriol succinate, conjugated estrogens including conjugated equine estrogens, and phytoestrogens.

Accordingly, in a more general aspect, the present invention also relates to a unit dosage form comprising a thin water-soluble film matrix, wherein
a) said film matrix comprises at least one water-soluble matrix polymer;
b) said film matrix comprises a compound exhibiting estrogenic activity in an amount corresponding to a therapeutically equivalent amount of 10-200 μg of estradiol; and
c) said film matrix has a thickness of less than 300 μm.

Accordingly, all statements made herein concerning preferred amounts of estradiol, excipients, etc., also apply to the above, and more general, aspect of the present invention.

### Manufacture

The unit dosage form of the invention may be prepared by standard methods well known to the pharmaceutical technologist.

Typically, a drug solution is prepared by dissolving the estradiol, or a derivative thereof such as estradiol hemihydrate or estradiol valerate, in an appropriate solvent. The solvent is preferably a relatively volatile solvent, such as an alcohol, in particular ethanol. A matrix polymer solution is then prepared by adding the water-soluble matrix polymer to a suitable solvent, such as alcohol, water or a mixture of an alcohol and water. Preferably, the solvent is an ethanol/water mixture. As will be understood, the time and conditions needed to dissolve the water-soluble matrix polymer will depend on the polymer and the solvent used. Thus, in some cases the water-soluble matrix polymer may dissolve easily at room temperature and with only gentle stirring, while in other cases it will be necessary to apply heat and vigorous stirring to the system. In a typical embodiment, the mixture is stirred for 1-4 hours, preferably for about 2 hours, or until a solution is obtained. The solution is typically stirred at a temperature of 60-80° C., such as about 70° C. After cooling to room temperature, the drug solution is poured into the matrix polymer solution and mixed thoroughly. The resulting solution (coating solution) can be used for coating immediately or within a few days. The various amounts of solvent, matrix polymer, etc. are adjusted to reach a solid content of the coating solution of about 5-50% by weight, preferably 10-40% by weight, in particular 20-35% by weight.

In an alternative embodiment, the coating solution may be prepared directly by adding the estradiol, or a derivative thereof such as estradiol hemihydrate or estradiol valerate, to an appropriate solvent, preferably an alcohol, in particular ethanol, followed by addition of water and subsequent addition of the matrix polymer. The mixture is then processed as described above until a solution is obtained. The resulting solution (coating solution) can be used for coating immediately or within a few days. The various amounts of solvent, matrix polymer, etc. are adjusted to reach a solid content of the coating solution of about 5-50% by weight, preferably 10-40% by weight, in particular 20-35% by weight.

Other excipients, auxiliary components and/or active drug substances may be added during any of the above mentioned steps.

If needed, the coating solution is degassed before being spread out on a suitable support or backing layer (liner). Examples of suitable liners include polyethylene-terephthalate (PET) liners, such as Perlasic® LF75 (available from Perlen Converting), Loparex® LF2000 (available from Loparex BV) and Scotchpack® 9742 (available from 3M Drug delivery Systems). In one embodiment of the invention, the coating solution is spread out with the aid of a spreading box onto a suitable liner and dried for 12-24 hours at room temperature. A thin transparent film is then produced, which is subsequently cut into pieces of the desired size and shape. Alternatively, the coating solution is coated as a thin film onto a suitable liner and in-line dried using an automated coating and drying equipment (e.g. by Coatema Coating Machinery GmbH, Dormagen, Germany) using a drying temperature of 40-125° C., such as 40-100° C. A thin transparent film is then produced, which is subsequently cut into pieces of the desired size and shape.

### Therapeutic Use and Administration

It will be understood that the unit dosage form of the invention is administered intraorally, i.e. the unit dosage form is administered to the oral cavity and the active drug substance is subsequently absorbed via one or more of the oral mucosae. Thus, the active drug substance is suitable for lingual administration, sublingual administration, buccal administration and palatal administration. Palatal or lingual administration, in particular lingual administration, is preferred.

According to this preferred embodiment the unit dosage form of the invention is simply applied to the tongue where it quickly dissolves, typically within 30 seconds, preferably within 15 seconds.

Accordingly, in another aspect, the present invention relates to a unit dosage form of the invention for use as a medicament.

In yet another aspect, the present invention relates to a unit dosage form of the invention for treating, alleviating or preventing a physical condition in a female mammal caused by insufficient endogenous levels of estrogen, such as osteoporosis, headaches, nausea, depression, vasomotor symptoms, symptoms of urogenital atrophy, decrease in bone mineral density or increased risk or incidence of bone fracture.

Deficient levels of estrogen can occur for a variety of reasons. For example, deficient levels of estrogen may be caused by e.g. natural menopause, peri-menopause, post-menopause, hypogonadism, castration or primary ovarian failure. Low levels of estrogen, irrespective of the cause, lead to an overall decreased quality of life for women. Symptoms, diseases and conditions range from merely being inconvenient to life threatening. The unit dosage form described herein provide effective alleviation of physiological and psychological signs of estrogen deficiency. Transient symptoms, such as vasomotor signs and psychological symptoms are certainly embodied with the realm of therapy.

Vasomotor symptoms comprise but are not limited to hot flushes, sweating attacks such as night sweats, and palpitations. The vasomotor symptoms may be “mild”, “moderate” or “severe” as, defined by the FDA guidelines (cited supra). Psychological symptoms of estrogen deficiency comprise, but are not limited to, insomnia and other sleep conditions, poor memory, loss of confidence, mood changes, anxiety, loss of libido, difficulties in concentration, difficulty in making decisions, diminished energy and drive, irritability and crying spells. The treatment or alleviation of the aforementioned symptoms can be associated with the peri-menopausal phase of a woman's life or after, sometimes long time after, menopause. It is anticipated that the unit dosage forms described herein are applicable to these and other transient symptoms during the peri-menopausal phase, menopause, or post-menopausal phase. Moreover, the aforementioned symptoms can be alleviated if the cause of the estrogen deficiency is hypogonadism, castration or primary ovarian failure. In another embodiment of the invention, the unit dosage forms described herein are used for the treatment or alleviation of permanent effects of estrogen deficiency. Permanent effects comprise physical changes such as urogenital atrophy, atrophy of the breasts, cardiovascular disease, changes in hair distribution, thickness of hair, changes in skin condition and osteoporosis. Urogenital atrophy, and conditions associated with it such as vaginal dryness, increase in vaginal pH and subsequent changes in flora, or events which lead to such atrophy, such as decreases in vascularity, fragmentation of elastic fibres, fusion of collagen fibres, or decreases in cell volume, are symptoms thought to be particularly relevant to be treated or alleviated with the unit dosage forms described herein. Furthermore, the unit dosage forms described herein are thought to be relevant to other urogenital changes associated with estrogen deficiency, decreases in mucus production, changes in cell population, decreases in glycogen production, decreases in growth of lactobacilli or increases in growth of streptococci, staphylococci, or coliform bacilli. Other associated changes that are thought to be preventable by administration of the unit dosage forms described herein are those that may render the vagina susceptible to injury or infection, such as exudative discharges, vaginitis, and dyspareunia. Furthermore, infections of the urinary tract and incontinence are other common symptoms associated with lowered estrogen levels. Other embodiments of the invention include the prevention or alleviation of physical changes associated with estrogen deficiency, such as changes in the skin, changes in hair distribution, thickness of hair, atrophy of the breasts, or, osteoporosis. The prevention and management of osteoporosis, most notably post-menopausal osteoporosis, is a particularly interesting embodiment of the invention. Furthermore, bone demineralisation, reduction of bone mass and density, thinning and interruption of trabeculae, and/or consequent increase in bone fractures or bone deformations are thought to be particularly relevant. The prophylactic treatment of osteoporosis is an interesting therapeutic application of the compositions of the invention. A particularly interesting embodiment of the invention is directed to lessening the frequency, persistence, duration and/or severity of hot flushes, sweating attacks, palpitations, sleep conditions, mood changes, nervousness, anxiety, poor memory, loss of confidence, loss of libido, poor concentration, diminished energy, diminished drive, irritability, urogenital atrophy, atrophy of the breasts, cardiovascular disease, changes in hair distribution, thickness of hair, changes in skin condition and osteoporosis (including prevention of osteoporosis), most notably hot flushes, sweating attacks, palpitations, sleep conditions, mood changes, nervousness, anxiety, urogenital atrophy, atrophy of the breasts, as well as prevention or management of osteoporosis. Another interesting embodiment of the invention is directed to treatment or alleviation of hot flushes, sweating attacks, palpitations, sleep conditions, mood changes, nervousness, anxiety, poor memory, loss of confidence, loss of libido, poor concentration, diminished energy, diminished drive, irritability, urogenital atrophy, atrophy of the breasts, cardiovascular disease, changes in hair distribution, thickness of hair, changes in skin condition and osteoporosis (including prevention of osteoporosis), most notably hot flushes, sweating attacks, palpitations, sleep conditions, mood changes, nervousness, anxiety, urogenital atrophy, atrophy of the breasts, as well as prevention or management of osteoporosis.

In a preferred embodiment of the invention, the female mammal to be treated according to the invention is a woman, in particular a postmenopausal woman.

The terms “pre-menopause”, “peri-menopause”, “menopause” and “post-menopause” are used in their conventional meaning, e.g. as defined on page 9 of “The Controversial Climateric”; P. A. van Keep et al. Ed., MTP Press (1981). More particularly, the term “menopause” is understood as the last natural (ovary-induced) menstruation. It is a single event and a result of an age-dependent dysfunction of the ovarian follicles. Menopause results from the ovaries decreasing their production of the sex hormones estrogen and progesterone. When the number of follicles falls below a certain threshold, the ovaries can no longer produce mature follicles and sex hormones. The ability to reproduce ends with menopause. The peri-menopausal phase begins with the onset of climacteric symptoms when the cycle becomes irregular and ends one year after menopause. The end of peri-menopausal phase can be identified after a protracted period of time without bleeding. Post-menopause is the phase that begins at menopause and continues until death.

In a further, and particular preferred embodiment of the invention, the postmenopausal woman to be treated according to the invention is a hysterectomised postmenopausal woman.

Hysterectomy is the surgical removal of the uterus. A total hysterectomy is removal of the uterus and cervix, A partial hysterectomy is removal of the uterus leaving the stump of the cervix (also called supra-cervical). Hysterectomy can be accompanied by surgical removal of the ovaries (oophorectomy). Removal of the female gonads, the ovaries, is female castration. Women who undergo total hysterectomy with bilateral salpingo-oophorectomy (removal of both ovaries, i.e. castration) lose most of their hormone production, including many estrogens and progestins. A woman who is undergoing natural menopause has intact and functional female organs, while a woman who has been hysterectomised and castrated does not. Accordingly, in the present context the term “hysterectomised woman” refers to a woman who has undergone total or partly hysterectomy.

It is well-established that exogenous estrogens stimulate the proliferation of the endometrium. In estrogen monotherapy, the opposing effect of progesterone, which terminates proliferation, is absent. The desquamation phase, during which the top layers of the endometrium are shed, does not occur and proliferation of the endometrium occurs to a greater extent than in the phases up to and including the pre-menopausal phase. The result is hyperplasia, a risk factor for endometrial cancer. Combination therapy, also referred to as opposed therapy, is a treatment where a progestin is added to protect the endometrium from hyperplasia. Accordingly, in another embodiment of the invention, in particular in connection with treatment, alleviation or prevention of diseases, conditions or symptoms associated with deficient endogenous levels of estrogen in a woman who has not undergone hysterectomy (a “non-hysterectomised woman”), a progestin may be incorporated in the unit dosage form of the invention in order to protect the endometrium from adverse effects caused by the exogenous estrogen. Alternatively, the progestin may be administered in a separate unit dosage form, such as an oral tablet. However, as explained above, it is believed that by administration of the doses of estradiol disclosed herein, in particular by administration of the “ultra low” or the “very low” doses of estradiol, the endometrium will not proliferate and, accordingly, co-administration of a progestin is not necessarily required.

Likewise, in another embodiment of the invention, in particular in connection with treatment, alleviation or prevention of diseases, conditions or symptoms associated with deficient endogenous levels of estrogen in a woman who has undergone hysterectomy (a “hysterectomised woman”), it is desirable that the unit dosage form does not contain a progestin. Thus, according to this embodiment, it is preferred that the unit dosage form of the invention contains the estradiol, or a hydrate of estradiol or a pharmaceutically acceptable ester of estradiol, as the only therapeutically active drug substance.

It is well-established that the average serum level of estradiol after oral administration of 2 mg estradiol is typically in the range of 80-100 μg/ml. This serum level is generally considered suitable for alleviating vasomotor symptoms, such as hot flushes. Furthermore, it is well-established that after oral administration, estradiol is extensively, metabolized during absorption and only about 5% of the estradiol becomes bioavailable (Kuhnt et. al. Drug Res. 1993; 43; 966-973).

As it appears from the experimental data provided in the examples, the unit dosage forms of the present invention have a considerable higher bioavailability than orally administered tablets. Thus, a bioavailability of more than 10%, such as more than 20%, e.g. more than 30% will typically be achieved. More particularly, a bioavailability in the range of from 10-90%, such as 20-80%, e.g. 30-80% will typically be achieved. In particular, if the therapeutically active substance is estradiol or estradiol hemihydrate, a bioavailability in the range of from 50-80%, usually 60-80% is achieved. This, in turn, has the consequence that therapeutic effective serum levels of estradiol can be achieved although a significantly lower dose of estradiol is administered as compared to oral administration. Likewise, if the therapeutically active susbstance is an estradiol ester, such as estradiol valerate, a bioavailability in the range of from 30-60%, usually 30-50% is achieved. Thus, also in this case is it possible to reach therapeutic effective serum levels of estradiol with a dose which is lower compared to oral administration. Thus, it is contemplated that by daily administration of the unit dosage forms described herein maximum serum levels (Cₘₐₓ) of estradiol in the range of from 100-1500 pg/ml, such as 150-1500 pg/ml can be achieved. More particularly, Cₘₐₓ values in the range of 500-800 pg/ml can be achieved by administration of 90 μg of estradiol and Cₘₐₓ values in the range of 100-250 pg/ml, preferably 150-250 pg/ml, can be achieved by administration of 118 μg of estradiol valerate. Furthermore, it is contemplated that administration of 150 μg estradiol will give rise to a Cₘₐₓ value in the range of 1000-1500 pg/ml.

Finally, it is the present inventors' experience that the serum estradiol values are somewhat dependent on the applied laboratory assay. Therefore, whenever reference is made to a certain serum estradiol concentration it should be understood that the concentration of estradiol is determined by the assay described in Example 4 herein.

The invention is further illustrated by the following, non-limiting, examples.

### EXPERIMENTAL

### Example 1

### Preparation of Wafers

### Preparation of the Coating Solution-Option A

A drug solution containing 0.558 g estradiol hemihydrate is prepared by dissolving the drug in 50 g ethanol (96%) under stirring. A polymer solution is prepared by strewing 149.442 g HPMC onto a mixture of 100 g ethanol (96%) and 150 g water. The HPMC dissolves after stirring for 2 hours at 70° C. After cooling to room temperature, the drug solution is poured into the polymer solution and mixed thoroughly. The resulting solution (coating solution) can be used for coating immediately or within a few days.

Preparation of the Coating Solution-Option B A coating solution is prepared by dissolving 0.558 g estradiol hemihydrate in 200 g ethanol (96%) under stirring. After admixing with 100 g water, 149.442 g HPMC is added and dissolves after stirring for 2 hours at 70° C. The resulting solution (coating solution) can be used for coating immediately or within a few days.

### Preparation of Wafers-Option 1

The coating solution is degassed and spread out with the aid of a spreading box onto a polyethylene-terephthalate (PET) liner (Perlasic® LF75) and dried for 24 hours at room temperature. A thin transparent film which is about 40 μm thick is produced. Wafers are obtained by cutting out samples of 5 cm² size.

### Preparation of Wafers-Option 2

The coating solution is degassed and coated as a thin film onto a polyethylene-terephthalate (PET) liner (Perlasic®LF75) and in-line dried using an automated coating and drying equipment (Coatema Coating Machinery GmbH, Dormagen, Germany). A drying temperature of 70° C. is applied. A thin transparent film which is about 40 μm thick is produced. Wafers are obtained by cutting out samples of 5 cm² size.

Using the above-mentioned manufacturing methods, wafers having the following composition were prepared:

Estradiol hemihydrate wafer, 30 μg (Formulation A)

| Name of ingredient | Quantity | Function |
|---|---|---|
| Active ingredients | | |
| Estradiol hemihydrate | 0.031 mg | Active |
| (~0.030 mg estradiol) | | ingredient |
| Other ingredients | | |
| HPMC | 24.969 mg | Matrix polymer |
| Purified water* | q.s. | Process solvent |
| Ethanol 96%* | q.s. | Process solvent |
| Total mass: | 25.000 mg | |

*evaporates during manufacturing

Estradiol hemihydrate wafer, 90 μg (Formulation B)

| Name of ingredient | Quantity | Function |
|---|---|---|
| Active ingredients | | |
| Estradiol hemihydrate | 0.093 mg | Active |
| (~0.090 mg estradiol) | | ingredient |
| Other ingredients | | |
| HPMC | 24.907 mg | Matrix polymer |
| Purified water* | q.s. | Process solvent |
| Ethanol 96%* | q.s. | Process solvent |
| Total mass: | 25.000 mg | |

*evaporates during manufacturing

Estradiol hemihydrate wafer, 270 μg (Formulation C)

| Name of ingredient | Quantity | Function |
|---|---|---|
| Active ingredients | | |
| Estradiol hemihydrate | 0.279 mg | Active |
| (~0.270 mg estradiol) | | ingredient |
| Other ingredients | | |
| HPMC | 24.721 mg | Matrix polymer |
| Purified water* | q.s. | Process solvent |
| Ethanol 96%* | q.s. | Process solvent |
| Total mass: | 25.000 mg | |

*evaporates during manufacturing

Estradiol valerate wafer, 30 μg (Formulation D)

| Name of ingredient | Quantity | Function |
|---|---|---|
| Active ingredients | | |
| Estradiol valerate | 0.039 mg | Active |
| (~0.030 mg estradiol) | | ingredient |
| Other ingredients | | |
| HPMC | 24.961 mg | Matrix polymer |
| Purified water* | q.s. | Process solvent |
| Ethanol 96%* | q.s. | Process solvent |
| Total mass: | 25.000 mg | |

*evaporates during manufacturing

Estradiol valerate wafer, 90 μg (Formulation E)

| Name of ingredient | Quantity | Function |
|---|---|---|
| Active ingredients | | |
| Estradiol valerate | 0.118 mg | Active |
| (~0.090 mg estradiol) | | ingredient |
| Other ingredients | | |
| HPMC | 24.882 mg | Matrix polymer |
| Purified water* | q.s. | Process solvent |
| Ethanol 96%* | q.s. | Process solvent |
| Total mass: | 25.000 mg | |

*evaporates during manufacturing

Estradiol valerate wafer, 270 μg (Formulation F)

| Name of ingredient | Quantity | Function |
|---|---|---|
| Active ingredients | | |
| Estradiol valerate | 0.353 mg | Active |
| (~0.270 mg estradiol) | | ingredient |
| Other ingredients | | |
| HPMC | 24.647 mg | Matrix polymer |
| Purified water* | q.s. | Process solvent |
| Ethanol 96%* | q.s. | Process solvent |
| Total mass: | 25.000 mg | |

*evaporates during manufacturing

Estradiol hemihydrate wafer, 40 μg (Formulation G)

| Name of ingredient | Quantity | Function |
|---|---|---|
| Active ingredients | | |
| Estradiol hemihydrate | 0.041 mg | Active |
| (~0.040 mg estradiol) | | ingredient |
| Other ingredients | | |
| HPMC | 24.959 mg | Matrix polymer |
| Purified water* | q.s. | Process solvent |
| Ethanol 96%* | q.s. | Process solvent |
| Total mass: | 25.000 mg | |

*evaporates during manufacturing

Estradiol hemihydrate wafer, 45 μg (Formulation H)

| Name of ingredient | Quantity | Function |
|---|---|---|
| Active ingredients | | |
| Estradiol hemihydrate | 0.047 mg | Active |
| (~0.045 mg estradiol) | | ingredient |
| Other ingredients | | |
| HPMC | 24.953 mg | Matrix polymer |
| Purified water* | q.s. | Process solvent |
| Ethanol 96%* | q.s. | Process solvent |
| Total mass: | 25.000 mg | |

*evaporates during manufacturing

Estradiol hemihydrate wafer, 50 μg (Formulation I)

| Name of ingredient | Quantity | Function |
|---|---|---|
| Active ingredients | | |
| Estradiol hemihydrate | 0.052 mg | Active |
| (~0.050 mg estradiol) | | ingredient |
| Other ingredients | | |
| HPMC | 24.948 mg | Matrix polymer |
| Purified water* | q.s. | Process solvent |
| Ethanol 96%* | q.s. | Process solvent |
| Total mass: | 25.000 mg | |

*evaporates during manufacturing

Estradiol hemihydrate wafer, 80 μg (Formulation J)

| Name of ingredient | Quantity | Function |
|---|---|---|
| Active ingredients | | |
| Estradiol hemihydrate | 0.083 mg | Active |
| (~0.080 mg estradiol) | | ingredient |
| Other ingredients | | |
| HPMC | 24.917 mg | Matrix polymer |
| Purified water* | q.s. | Process solvent |
| Ethanol 96%* | q.s. | Process solvent |
| Total mass: | 25.000 mg | |

*evaporates during manufacturing

Estradiol hemihydrate wafer, 120 μg (Formulation K)

| Name of ingredient | Quantity | Function |
|---|---|---|
| Active ingredients | | |
| Estradiol hemihydrate | 0.124 mg | Active |
| (~0.120 mg estradiol) | | ingredient |
| Other ingredients | | |
| HPMC | 24.876 mg | Matrix polymer |
| Purified water* | q.s. | Process solvent |
| Ethanol 96%* | q.s. | Process solvent |
| Total mass: | 25.000 mg | |

*evaporates during manufacturing

Estradiol hemihydrate wafer, 150 μg (Formulation L)

| Name of ingredient | Quantity | Function |
|---|---|---|
| Active ingredients | | |
| Estradiol hemihydrate | 0.155 mg | Active |
| (~0.150 mg estradiol) | | ingredient |
| Other ingredients | | |
| HPMC | 24.845 mg | Matrix polymer |
| Purified water* | q.s. | Process solvent |
| Ethanol 96%* | q.s. | Process solvent |
| Total mass: | 25.000 mg | |

*evaporates during manufacturing

As will be understood, analogous wafers which contain other amounts of estradiol and/or which contain estradiol derivatives can easily be manufactured using the procedures described herein. In preferred embodiments, sweeteners and/or flavours are added to the formulations.

### Example 2

### Clinical Study (PK Study)

Study outline

| | |
|---|---|
| Centres and countries | 1 centre in EU |
| Study objective | To investigate the relative bioavailability and the |
| | PK profile of estradiol after application of |
| | different wafers in comparison with the |
| | application of estrogens administered via other |
| | routes of application |
| Study design | Single center, open-label, randomised crossover |
| | study |
| Study population | 11 healthy postmenopausal women |
| Treatment | Two different estradiol wafer formulations (B |
| | and E) |
| | One oral estradiol tablet (Avaden ®, 1 mg) |
| | One intranasal estradiol spray |
| | (Aerodiol ®, 300 μg) |
| Duration | Up to four weeks as four single-dose treatments |
| | with one week washout between each treatment |
| Variables | The dose-normalised AUC(0-tₗₐₛₜ) of estradiol |
| | after application of a wafer formulation in |
| | comparison with an oral tablet and an intranasal |
| | spray. |
| | Secondary variable: |
| | Descriptive statistics of all pharmacokinetic |
| | parameters like Cₘₐₓ, tₘₐₓ, AUC(0-tₗₐₛₜ), etc. |

### Results

The following not-dose-normalised data, where all parameters are expressed in percentage of the respective Aerodiol® values, were obtained:

| | Dose* | AUC | Cₘₐₓ |
|---|---|---|---|
| Formulation | (%) | (%) | (%) |
| Wafer B (estradiol hemihydrate) | 30 | 60 | 45 |
| Wafer E (estradiol valerate) | 30 | 32 | 12 |
| Aerodiol ® (intranasal) | 100 | 100 | 100 |
| Avaden ® (oral) | 333 | 43 | 2 |

*calculated as the corresponding amount of estradiol

The above data show that a “puls-like” pharmacokinetic profile was obtained, and that the bioavailability (and Cₘₐₓ) of the dosage forms of the invention, in particular dosage forms comprising estradiol hemihydrate, is significantly higher than the bioavailability (and Cₘₐₓ) for orally administered tablets.

Furthermore, a dosage form of the invention which comprises only 30% of the Aerodiol® dose still gave rise to a bioavailability which was 60% of the Aerodiol® value. Accordingly, the estradiol, when formulated according to the invention, appears to have a bioavailability which is even higher than if administered intranasally. More particularly, the dosage forms of the present invention have a bioavailability which is twice the bioavailability of intranasally administered formulations. Consequently, only half the dose is needed in the formulations according to the invention compared to the dose needed if administered intranasally.

### Example 3

### Clinical Trials

Study outline

| | |
|---|---|
| Centres and countries | >90 centres |
| | US: approximately 50% of centres/patients |
| | Other regions: EU |
| Study objective | To investigate efficacy (reduction of moderate to |
| | severe hot flushes) and general safety of five |
| | different strength estradiol wafer formulations in |
| | comparison to placebo and comparator |
| Study design | Multicenter, double-blind, randomised, placebo- |
| | controlled, active-controlled study |
| Study population | hysterectomised postmenopausal women |
| Treatment | Five estradiol wafer arms |
| | Placebo |
| | Comparator (1-2 doses of Premarin ®) |
| Duration | 13 cycles (12 months) |
| Variables | The primary efficacy variable is the mean |
| | change of moderate to severe host flushes from |
| | baseline at weeks 4 and 12 as defined in the |
| | FDA guidelines (cited supra) |
| | Secondary variable: |
| | Symptoms of vulvar and vaginal atrophy |
| | Laboratory measurements of lipids, |
| | coagulation and other variables of liver |
| | estrogenicity (e.g. SHBG, IGF1) |
| | Bone turnover markers |
| | Mammographic breast density |
| | General safety: Adverse events, compliance, |
| | general physical and gynecological |
| | examination (incl. cervical smear), vital signs |
| | and body weight |

Efficacy and safety for a new pharmaceutical drug preparation is commonly investigated in a clinical phase 3 study. Recommendations of the US and EU authorities (FDA guidelines (cited supra), and Guideline on clinical investigation of medical products for hormone replacement therapy of oestrogen deficiency symptoms in postmenopausal women; EMEA; The European Medicines Agency; October 2005) on how to design a study for the indication vasomotor symptoms (hot flushes) in postmenopausal women include for example:

The study should be conducted in a randomised, double-blind, placebo-controlled design for a treatment duration of 12 weeks. As a main prerequisite patients should show a pre-defined minimum number of hot flushes per day before they start into the treatment phase (at baseline) of the study. Only moderate to severe hot flushes are categorised to require treatment.

The patients receive a diary specifically designed for the documentation of hot flushes which serves as source data in the study periods before and during the treatment phase. Women are to record daily the number of hot flushes they experience and the severity thereof:

Mild: Sensation of heat without sweating
Moderate: Sensation of heat with sweating; able to continue activity
Severe: Sensation of heat with sweating; causing cessation of activity

The effectiveness of the study preparation will be shown by a reduction of frequency and severity of moderate to severe hot flushes at week 12. The mean change of hot flushes from baseline to week 12 and the mean change at week 12 of the study preparation versus placebo should show statistically significance. Assuming a responder rate (responder: defined as at least 75% reduction in moderate to severe hot flushes from baseline at week 12) of 25% for placebo, the application of an estradiol-containing wafer would be regarded effective in the treatment of hot flushes in case the lowest effective estradiol dose would show a responder rate of approximately 45%; a responder rate of approximately 90% would reflect the efficacy of the standard dose used in the market (equivalent to e.g. 1 mg estradiol p.o.) and a responder rate of >90% would reflect accordingly higher doses; medium effective doses would result in responder rates between the lowest effective dose and standard dose. If the responder rate for placebo would result in a lower or higher percentage (as assumed above), the rates assumed for the study preparations would have to be adjusted accordingly.

### Example 4

### Estradiol Assays

Estradiol may be determined by two different assays:

### GC/MS/MS

The analytes and their deuterated internal standards are extracted from 1.00 ml of human serum using BondElut Certify® solid-phase cartridges. Estradiol and estrone are eluted from the solid phase cartridges with ethyl acetate. The analytes undergo three separate derivatisation steps: (1) reaction with pentafluorobenzoyl chloride, (2) reaction with O-(2,3,4,5,6-pentafluorobenzyl)-hydroxylamine hydrochloride, and (3) reaction with MSTFA. The derivatised analytes are then separated by gas chromatography using a DB-17 fused silica capillary column and detected by tandem mass spectrometry using negative ion chemical ionization. A 1/(concentration)² weighted least-squares regression procedure is used to fit a linear function to the calibration data.

### LC/MS/MS

A 500 μl sample aliquot is fortified with 25 μl of internal standard working solution. Analytes are isolated through liquid-liquid extraction with 5.0 ml of 10:90 (v/v) ethyl acetate/hexane. The solvent is evaporated under a stream of nitrogen at 40-50° C. and the remaining residue is derivatizsd. The derivatised analytes are extracted into 3.0 m of 10:90 (v/v) ethyl acetate/hexane, the solvent is evaporated, and the remaining residue is reconstituted with 150 μl acetonitrile and 200 μl water. The final extract is analyzed via high-performance liquid chromatography with tandem mass spectrometry detection.

1. A unit dosage form comprising a thin water-soluble film matrix, wherein
a) said film matrix comprises at least one water-soluble matrix polymer;
b) said film matrix comprises 10-200 μg of estradiol, or a therapeutically equivalent amount of a hydrate of estradiol or a therapeutically equivalent amount of a pharmaceutically acceptable ester of estradiol; and
c) said film matrix has a thickness of less than 300 μm.

2. The dosage form according to claim 1, wherein said water-soluble matrix polymer is selected from the group consisting of a cellulosic material, a synthetic polymer, a gum, a protein, a starch, a glucan and mixtures thereof.

3. The dosage form according to claim 2, wherein said cellulosic material is selected from the group consisting of carboxymethyl cellulose, methyl cellulose, ethyl cellulose, hydroxymethyl cellulose, hydroxyethyl cellulose, hydroxypropyl cellulose, hydroxymethylpropyl cellulose and hydroxypropylmethyl cellulose.

4. The dosage form according to claim 3, wherein said cellulosic material is hydroxypropylmethyl cellulose or hydroxypropyl cellulose.

5. The dosage form according to claim 4, wherein said cellulosic material is hydroxypropylmethyl cellulose.

6. The dosage form according to claim 1, wherein said hydrate of estradiol is estradiol hemihydrate.

7. The dosage form according to claim 1, wherein said pharmaceutically acceptable ester of estradiol is selected from the group consisting of estradiol valerate, estradiol acetate, estradiol propionate, estradiol enantate, estradiol undecylate, estradiol benzoate, estradiol cypionate, estradiol sulfate and estradiol sulfamate.

8. The dosage form according to claim 7, wherein said pharmaceutically acceptable ester of estradiol is estradiol valerate.

9. The dosage form according to claim 1, wherein said film matrix comprises 10-60 μg of estradiol, or a therapeutically equivalent amount of a hydrate of estradiol or a therapeutically equivalent amount of a pharmaceutically acceptable ester of estradiol.

10. The dosage form according to claim 1, wherein said film matrix comprises >60-200 μg of estradiol, or a therapeutically equivalent amount of a hydrate of estradiol or a therapeutically equivalent amount of a pharmaceutically acceptable ester of estradiol.

11. The dosage form according to claim 1, wherein said film matrix has a thickness of less than 200 μm.

12. The dosage form according to claim 11, wherein said film matrix has a thickness of less than 100 μm.

13. The dosage form according to claim 1, wherein said film matrix has a surface area of 2-10 cm².

14. The dosage form according to claim 13, wherein said film matrix has a surface area of 3-7 cm².

15. The dosage form according to claim 14, wherein said film matrix has a surface area of 4-6 cm².

16. The dosage form according to claim 1, wherein said film matrix has a weight in the range of from 5-200 mg.

17. The dosage form according to claim 16, wherein said film matrix has a weight in the range of from 10-100 mg.

18. The dosage form according to claim 17, wherein said film matrix has a weight in the range of from 10-50 mg.

19. The dosage form according to claim 1, wherein said estradiol, or hydrate of estradiol or pharmaceutically acceptable ester of estradiol, is the only therapeutically active drug substance present in said unit dosage form.

20. The dosage form according to claim 1, wherein said unit dosage form does not contain a progestin.

21. The dosage form according to claim 1, wherein said film matrix further comprises a progestin.

22. A unit dosage form as defined in claim 1, for use as a medicament.

23. A unit dosage form as defined in claim 1, for treating, alleviating or preventing a physical condition in a female mammal caused by insufficient endogenous levels of estrogen.

24. The dosage form according to claim 23, wherein said physical condition is selected from the group consisting of osteoporosis, headaches, nausea, depression, vasomotor symptoms, symptoms of urogenital atrophy, decrease in bone mineral density, and increased risk or incidence of bone fracture.

25. The dosage form according to claim 24, wherein said vasomotor symptoms are selected from the group consisting of hot flushes, sweating attacks including night sweats, and palpitations.

26. The dosage form according to claim 23, wherein said female mammal is a postmenopausal woman.

27. A dosage form as defined in claim 1 for treating, alleviating or preventing a physical condition in a hysterectomised postmenopausal woman caused by insufficient endogenous levels of estrogen.

28. A dosage form as defined in claim 21 for treating, alleviating or preventing a physical condition in a non-hysterectomised postmenopausal woman caused by insufficient endogenous levels of estrogen.

29. A method for treating, alleviating or preventing a physical condition in a female mammal caused by insufficient endogenous levels of estrogen, the method comprising administering a unit dosage form as defined in claim 1 to a female mammal in need thereof.

30. The method according to claim 29, wherein said physical condition is selected from the group consisting of osteoporosis, headaches, nausea, depression, vasomotor symptoms, symptoms of urogenital atrophy, decrease in bone mineral density, and increased risk or incidence of bone fracture.

31. The method according to claim 30, wherein said vasomotor symptoms are selected from the group consisting of hot flushes, sweating attacks including night sweats, and palpitations.

32. The method according to claim 29, wherein said female mammal is a postmenopausal woman.

33. A method for treating, alleviating or preventing a physical condition in a hysterectomised postmenopausal woman caused by insufficient endogenous levels of estrogen, the method comprising administering a unit dosage form as defined in claim 1 to a hysterectomised postmenopausal woman in need thereof.

34. A method for treating, alleviating or preventing a physical condition in a non-hysterectomised postmenopausal woman caused by insufficient endogenous levels of estrogen, the method comprising administering a unit dosage form as defined in claim 21 to a non-hysterectomised postmenopausal woman in need thereof.
